(19) United States
(12) Patent Application Publication
(10) Pub. No.: US 2021/0267911 A1
(43) Pub. Date: Sep. 2, 2021

(54) COMPOSITIONS AND METHODS FOR ADDRESSING VISION IMPAIRMENT

(71) Applicant: Script Essentials, LLC, Broomfield, CO (US)

(72) Inventor: Suzy Cohen, Broomfield, CO (US)

(21) Appl. No.: 17/099,077

(22) Filed: Nov. 16, 2020

Related U.S. Application Data
(60) Provisional application No. 62/982,381, filed on Feb. 27, 2020.

(51) Int. Cl. A61K 31/07; A61K 31/375; A61K 31/355; A61K 33/30; A61K 36/185; A61K 36/88; A61K 31/05; A61K 9/00; A61K 47/46

(52) U.S. Cl. CPC A61K 31/07; A61K 31/375; A61K 31/355; A61K 33/30; A61K 47/46; A61K 36/88; A61K 31/05; A61K 9/0056; A61K 36/185

(57) ABSTRACT

The present disclosure relates to compositions for improving vision in a patient and methods of administering a composition to a patient, particularly in the form of a dietary supplement, which addresses vision impairment and promotes the health of a patient experiencing problems associated with deteriorated eyesight, including macular degeneration. The present disclosure also relates to methods for formulating the composition described herein.

### CROSS REFERENCE TO RELATED APPLICATIONS

This application claims priority to and the benefit of, pursuant to 35 U.S.C. § 119(e), U.S. Provisional Patent Application No. 62/982,381, filed on Feb. 27, 2020, which is incorporated by reference herein in its entirety.

### FIELD OF THE INVENTION

The present invention is directed to compounds, particularly dietary supplements, used for improving eye function, addressing vision impairment and/or treating an individual subject suffering from vision impairment. The present invention is also directed to methods for treatment of a patient experiencing poor vision or related problems with eyesight, including but not limited to macular degeneration, as well as methods for formulating and administering a supplement to address the same.

### BACKGROUND OF THE INVENTION

The frequency of macular degeneration in persons over the age of 55 has increased in recent years and is now considered the leading cause of loss of eyesight. The cause of macular degeneration is unknown and remains unresolved despite attempts to improve the condition known in the art. Another trend among aging populations in the United States and throughout the world is the development of cataracts and related impairment to visual acuity. With the increased use of mobile electronic devices, tablets, laptops, etc. the strain on a human's eyes today may cause other problems, including blurry vision, dry eyes, presbyopia, and further serve to amplify age-related vision problems such as macular degeneration, cataracts and temporal arteritis.

Further, the dramatic increase in persons with type 2 diabetes (formerly known as non-insulin-dependent diabetes mellitus (NIDDM) and also known as Adult Onset Diabetes) may have a profound impact on human vision, including diabetic retinopathy, diabetic macular edema, cataracts, and glaucoma. Over time, diabetes can cause damage to an individual's eyes, leading to even blindness. The estimated number of individuals with diagnosed and yet-to-be diagnosed diabetes is approximately 29.1 million people in the United States alone, which equates to approximately 9.3 percent of the population.

Accordingly, there is a long-felt but unresolved need to address these and other problems, which are the subject of the compositions and methods described in detail herein.

### SUMMARY OF THE INVENTION

The invention in embodiments relates to a compound, particularly in the form of a dietary supplement, which addresses vision impairment and the shortcomings in the art with respect to improved eye function and enhancing healthy vision. As disclosed in more detail in the Detailed Description, the present invention provides compositions and methods for treating a person with vision impairment, blurry vision, dry eyes, presbyopia, macular edema, cataracts, glaucoma, macular degeneration and temporal arteritis, among other conditions. Methods for forming the compound are further described herein.

Through experimentation it has been found that including various nutrients can be beneficial to people who have conditions that adversely affect their vision. More particularly, people who are deficient in vitamin C may have increased risk of suffering from one of the conditions described above. Vitamin C may also reduce damage to tissue caused by UV radiation. Individuals who become deficient in vitamin C (because it is not manufactured in the body) may suffer from numerous conditions, including premature signs of aging, joint pain, autoimmune conditions, atherosclerosis and digestive disorders. Other signs of deficiency may include cardiovascular disorders, easy bruising, and swollen and bleeding gums. Therefore, providing a composition that also comprises appropriate levels of vitamin C to counteract these problems would be beneficial.

In embodiments, the unique combination of the composition is preferably administered orally in the form of a capsule, tablet, powder or lozenge. The unique combination has synergistic advantages over previously known compositions, all proportioned to provide the most benefit to people affected by the problems described above.

The composition is preferably comprised of a unique and novel formulation in predetermined amounts, and further provides benefits previously unexpected. In a preferred embodiment, the composition is comprised of Vitamin A (as retinyl palmitate), Vitamin C (as ascorbic acid), Vitamin E (as mixed tocopherols), Zinc (as zinc glycinate), Black Currant 25% extract, Saffron EX 5C, Lutein and Zeaxanthin, or combinations/sub-combinations thereof.

In another preferred embodiment, the composition comprises the following, with variability in dosages listed below:

1) Vitamin A (as retinyl palmitate) between about 500 IU to 50,000 IU;

2) Vitamin C (as ascorbic acid)—between about 10 mg and about 2,000 mg;

3) Vitamin E—(as mixed tocopherols) between about 10 mg and about 1,000 mg;

4) Zinc—(as zinc glycinate) between about 1 mg and about 50 mg;

5) Black Currant 25% Extract—between about 10 mg and 1,000 mg;

6) Saffron EX 5C—between about 2 mg and 60 mg;

7) Lutein—between about 1 mg and about 100 mg; and

8) Zeaxanthin—between about 0.2 mg and about 10 mg.

In a most preferred embodiment, the composition comprises the following combination having the synergistic qualities described herein, in the quantities listed below:

1) about 10,000 IU of Vitamin A, preferably as retinyl palmite;

2) about 100 mg of Vitamin C, preferably as ascorbic acid;

3) about 100 mg of Vitamin E, preferably as mixed tocopherols;

4) about 10 mg of Zinc, preferably as zinc glycinate;

5) about 100 mg of Black Currant 25% Extract;

6) about 20 mg of Saffron EX 5C;

7) about 10 mg of Lutein; and

8) about 2 mg of Zeaxanthin.

In one embodiment, the composition is provided as a dietary supplement. In one embodiment, the composition is administered in the form of a vegetable-based capsule, and two capsules are administered daily. In another embodiment, the composition is administered in the form of a powder, a gummy chew, a tablet, a lozenge or a liquid extract. In a preferred embodiment, the formulation is unflavored, but in further embodiments, the composition may contain one or more palatability agents to favorably alter the taste of the composition for human consumption.

Methods for treatment of individuals with vision impairment, including but not limited to any of the disorders listed above, are also expressly made a part of this disclosure. Methods for formulating and administering the supplement described herein are also within the scope of the present disclosure.

It is to be expressly understood that he above-described embodiments, objectives, and configurations are neither complete nor exhaustive. The Summary of the Invention is neither intended, nor should it be construed as being representative of the full extent and scope of the present invention. Other advantages will be apparent from the disclosure of the invention(s) contained herein.

Embodiments of the present invention are set forth in various levels of detail in the Summary of the Invention, and no limitation as to the scope of the present invention is intended by either the inclusion or non-inclusion of elements in this Summary of the Invention. Additional aspects of the present invention will be readily apparent from the view of one of ordinary skill in the art.

### DETAILED DESCRIPTION

The following Detailed Description sets forth numerous different embodiments and examples of compositions and methods of treatment. Accordingly, it should be understood that the legal scope of the description is defined by the words of the claims set forth at the end of this disclosure. The Detailed Description is to be construed as exemplary only. The following description does not describe every possible embodiment, since describing every possible embodiment would be impractical, if not impossible. Numerous alternative embodiments could be implemented, using either current technology or technology developed after the filing date of this patent, which would be encompassed by the scope of the claims.

As used herein, references to “the present invention” or aspects thereof should be understood to mean certain embodiments of the present invention and should not necessarily be construed as limiting all embodiments to a particular description.

As used herein, the phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together.

Unless otherwise indicated, all numbers expressing quantities, amounts, dimensions, conditions, and so forth used in the specification and claims are to be understood as being modified in all instances by the term “about”.

The term “a” or “an” entity, as used herein, refers to one or more of that entity. As such, the terms “a” (or “an”), “one or more” and “at least one” can be used interchangeably herein.

The use of “including,” “comprising,” or “having” and variations thereof herein is meant to encompass the items listed thereafter and equivalents thereof as well as additional items. Accordingly, the terms “including,” “comprising,” or “having” and variations thereof can be used interchangeably herein.

It shall be understood that the term “means” as used herein shall be given its broadest possible interpretation in accordance with 35 U.S.C. § 112(f). Accordingly, a claim incorporating the term “means” shall cover all structures, materials, or acts set forth herein, and all of the equivalents thereof. Further, the structures, materials, or acts and the equivalents thereof shall include all those described in the summary of the invention, brief description of the drawings, detailed description, abstract, and claims themselves.

In varying embodiments described herein, the present invention relates to a compound that improves healthy eye function. Certain elements of the novel compounds and methods for formulating the same are described in varying levels of detail herein.

Composition:

In a most preferred embodiment, the composition comprises the following combination of elements having the synergistic qualities described herein:

1) about 10,000 IU of Vitamin A, preferably as retinyl palmitate;

2) about 100 mg of Vitamin C, preferably as ascorbic acid;

3) about 100 mg of Vitamin E, preferably as mixed tocopherols;

4) about 10 mg of Zinc, preferably as zinc glycinate;

5) about 100 mg of Black Currant 25% Extract;

6) about 20 mg of Saffron EX 5C;

7) about 10 mg of Lutein; and

8) about 2 mg of Zeaxanthin.

Each of the foregoing is described in more detail below.

Vitamin A

In a preferred embodiment, the composition comprises Vitamin A, preferably in the form of retinyl palmitate, which has been discovered through experimentation to have synergistic qualities when combined with one or more additional elements described in this Detailed Description. In a preferred embodiment, vitamin A is provided to a patient suffering from impaired or unhealthy eye functioning in a dosage ranging from about 500 IU to 50,000 IU. In a most preferred embodiment, the composition comprises about 10,000 IU of Vitamin A (as retinyl palmitate).

Vitamin C

In a preferred embodiment, the compound is comprised of a pre-determined amount of vitamin C. Vitamin C, also known as ascorbic acid, is a water-soluble vitamin and an important cofactor in at least eight enzymatic reactions, which through experimentation have been found to support healthy vision and provide anti-aging properties. These enzymatic reactions are important, and increasing evidence gathered during experimentation indicates vitamin C improves the absorption of minerals, most notably iron, in adults and children.

Furthermore, vitamin C is essential to wound healing and in the creation of skin, tendons, ligaments, and blood vessels of the human eye and in other regions of the human anatomy.

In a preferred embodiment, the composition comprises Vitamin C, preferably as ascorbic acid, which has been discovered through experimentation to have synergistic qualities when combined with one or more additional elements described in this Detailed Description. In a preferred embodiment, vitamin C is provided to a patient suffering from impaired or unhealthy eye functioning in a dosage ranging from about 10 mg and about 2,000 mg. In a most preferred embodiment, the composition comprises about 100 mg of Vitamin C as ascorbic acid.

Vitamin E

Vitamin E has also been found to improve healthy tissue and eye function. In a preferred embodiment, the composition comprises Vitamin E, preferably as mixed tocopherols, which has been discovered through experimentation to have synergistic qualities when combined with one or more additional elements described in this Detailed Description. In a preferred embodiment, vitamin E is provided to a patient suffering from impaired or unhealthy eye functioning in a dosage ranging from about 10 mg and about 1,000 mg. In a most preferred embodiment, the composition comprises about 100 mg of Vitamin E as mixed tocopherols.

Zinc

In a preferred embodiment, the composition comprises Zinc, preferably as zinc glycinate, which has been discovered through experimentation to have synergistic qualities when combined with one or more additional elements described in this Detailed Description.

In a preferred embodiment, Zinc is provided to a patient suffering from impaired or unhealthy eye functioning in a dosage ranging from about 1 mg and about 50 mg. In a most preferred embodiment, the composition comprises about 10 mg of Zinc as zinc glycinate.

Black Currant In a preferred embodiment, the composition comprises Black Currant 25% Extract, which has been discovered through experimentation to have synergistic qualities when combined with one or more additional elements described in this Detailed Description. In a preferred embodiment, Black Currant 25% Extract is provided to a patient suffering from impaired or unhealthy eye functioning in a dosage ranging from about 10 mg and 1,000 mg. In a most preferred embodiment, the composition comprises about 100 mg of Black Currant 25% Extract.

Saffron

In a preferred embodiment, the composition comprises Saffron EX 5C, which has been discovered through experimentation to have synergistic qualities when combined with one or more additional elements described in this Detailed Description. In a preferred embodiment, Saffron EX 5C is provided to a patient suffering from impaired or unhealthy eye functioning in a dosage ranging from about 2 mg and 60 mg. In a most preferred embodiment, the composition comprises about 20 mg of Saffron EX 5C.

Lutein

In a preferred embodiment, the composition comprises between about 1 mg and about 100 mg of Lutein, which has been discovered through experimentation to have synergistic qualities when combined with one or more additional elements described in this Detailed Description. In a most preferred embodiment, the composition comprises about 10 mg of Lutein.

Zeaxanthin

In a preferred embodiment, the composition comprises Zeaxanthin, which has been discovered through experimentation to have synergistic qualities when combined with one or more additional elements described in this Detailed Description. In a preferred embodiment, Zeaxanthin is provided to a patient suffering from impaired or unhealthy eye functioning in a dosage ranging from about 0.2 mg and about 10 mg. In a most preferred embodiment, the composition comprises about 2 mg of Zeaxanthin.

Absent Elements

In addition, the composition of a preferred embodiment is substantially free of the following common allergens: gluten, wheat, eggs, peanuts, tree nuts, dairy, sugar and fish/shellfish. The composition preferably does not contain artificial colors, flavors, or preservatives, and is free from magnesium stearate (a common lubricant used in the manufacture of pharmaceuticals and dietary supplements).

Additional Elements

In varying embodiments, the composition can further comprise variances, particularly with respect to encapsulation and/or powder formulations. According to certain embodiments, the compositions described herein can further be provided with one or more palatability agents. These palatability agents serve to add flavor to the composition so that an effective dosage is easier to be ingested. It is within the scope of the present invention that any safe, flavor enhancing palatability agent can be used in a composition of the present invention. Particularly suitable palatability agents for use in the composition of the present invention include, but are not limited to, plant oils, plant hydrolysates, yeast, yeast hydrolysates, and combinations thereof.

Methods

Another aspect of the present invention is a method to treat a patient experiencing vision impairment, blurry vision, dry eyes, presbyopia, macular edema, cataracts, glaucoma, macular degeneration and temporal arteritis, among other conditions. In embodiments, the method of treatment comprises administering a composition to a patient in need thereof, the composition comprising a unique combination and blend of Vitamin A (as retinyl palmitate), Vitamin C (as ascorbic acid), Vitamin E (as mixed tocopherols), Zinc (as zinc glycinate), Black Currant 25% extract, Saffron EX 5C, Lutein and Zeaxanthin, or combinations/sub-combinations thereof. In a preferred embodiment, the method of improving vision in a patient exhibiting impairment or degeneration relating to the same comprises: administering a composition consisting essentially of: about 10,000 IU of Vitamin A; about 100 mg of Vitamin C; about 100 mg of Vitamin E; about 10 mg of Zinc; about 100 mg of Black Currant 25% Extract; about 20 mg of Saffron EX 5C; about 10 mg of Lutein; and about 2 mg of Zeaxanthin.

According to one embodiment, during the method the patient is treated by providing an effective amount of the composition. In certain embodiments, the composition can be taken by the patient daily, without regard to meals or time of day ingested. In one such embodiment, the composition is administered in the form of a capsule or two capsules that are fit for human consumption. In other embodiments, up to four capsules are administered daily to the patient. In another embodiment, the composition is administered in the form of a tablet or up to four tablets on a daily basis.

Other components such as a palatability agent can also be added to the composition. In a preferred embodiment, about 1 and 4 capsules or tablets of the composition can be provided to the patient daily. The composition can be provided in the form of a capsule, a tablet, a suspension, a sublingual spray, a powder, or a lozenge. The capsule can be size 0, which corresponds to an overall closed length of between about 20.7-21.7 mm, and an average weight of between 98-106.4 mg +/−6%. The number of capsules can be sized to provide the patient depending on the characteristics of the patient and the degree of degeneration the patient is experiencing, by way of example. As the number varies, the size of the capsule can vary as well.

According to another embodiment, a method to prepare a compound is disclosed. The components are mixed in a proprietary blend and may be provided in a delivery device, for example, in the form of a capsule, tablet, lozenge or powder.

While various embodiments of the present disclosure have been described in detail, it is apparent that modifications and alterations of those embodiments will occur to those skilled in the art. However, it is to be expressly understood that such modifications and alterations are within the scope and spirit of the present disclosure, as set forth in the following claims. Ranges have been discussed and used within the forgoing description. One of skill in the art will understand that any sub-range within the stated range would be suitable, as would any number within the broad range, without deviating from the spirit of the present invention.

The foregoing discussion of the disclosure has been presented for purposes of illustration and description. The foregoing is not intended to limit the disclosure to the form or forms disclosed herein. In the foregoing Detailed Description for example, various features of the disclosure are grouped together in one or more embodiments for the purpose of streamlining the disclosure. This method of disclosure is not to be interpreted as reflecting an intention that the claimed disclosure requires more features than are expressly recited in each claim. Rather, as the following claims reflect, inventive aspects lie in less than all features of a single foregoing disclosed embodiment.

What is claimed is:

1. A method of treating a patient experiencing vision impairment or degeneration, comprising:
administering to the patient a composition comprising:
between about 500 IU and about 50,000 IU of Vitamin A;
between about 10 mg and about 2,000 mg of Vitamin C;
between about 10 mg and about 1,000 mg of Vitamin E;
between about 1 mg and about 50 mg of Zinc;
between about 10 mg and about 1,000 mg of Black Currant 25% Extract;
between about 2 mg and about 60 mg of Saffron EX 5C;
between about 1 mg and about 100 mg of Lutein; and
between about 0.2 mg and about 10 mg of Zeaxanthin.

2. The method of claim 1, wherein the vision impairment or degeneration comprises one or more of blurry vision, dry eyes, presbyopia, macular edema, cataracts, glaucoma, macular degeneration and temporal arteritis.

3. The method of claim 1, wherein the composition is administered to the patient in the form of a dietary supplement.

4. The method of claim 1, wherein the composition is administered to the patient in the form of a capsule or tablet.

5. The method of claim 1, wherein the Vitamin A is in the form of retinyl palmitate.

6. The method of claim 1, wherein the Vitamin C is in the form of ascorbic acid.

7. The method of claim 1, wherein the Vitamin E is in the form of mixed tocopherols.

8. The method of claim 1, wherein the Zinc is in the form of Zinc Glycinate.

9. The method of claim 1, wherein the composition administered to the patient further comprises a palatability agent selected from one or more of a plant oil, plant hydrolysates, yeast, yeast hydrolysates, and combinations thereof.

10. The method of claim 1, wherein the composition administered to the patient does not include milk, casein, gluten, wheat, eggs, peanuts, tree nuts, dairy, corn, soybeans, sugar and fish/shellfish.

11. A method of treating the vision of a patient exhibiting impairment or degeneration relating to the same, wherein the method comprises administering to the patient a composition consisting essentially of:
about 10,000 IU of Vitamin A;
about 100 mg of Vitamin C;
about 100 mg of Vitamin E;
about 10 mg of Zinc;
about 100 mg of Black Currant 25% Extract;
about 20 mg of Saffron EX 5C;
about 10 mg of Lutein; and
about 2 mg of Zeaxanthin.

12. The method of claim 11, wherein the Vitamin A is in the form of retinyl palmitate.

13. The method of claim 11, wherein the Vitamin C is in the form of ascorbic acid.

14. The method of claim 11, wherein the Vitamin E is in the form of mixed tocopherols.

15. The method of claim 11, wherein the Zinc is in the form of Zinc Glycinate.

16. The method of claim 11, wherein the composition administered to the patient is devoid of the following: milk, casein, gluten, wheat, eggs, peanuts, tree nuts, dairy, corn, soybeans, sugar and fish/shellfish.

17. The method of claim 11, wherein the composition is administered to the patient in the form of a dietary supplement.

18. The method of claim 11, wherein the composition is administered to the patient in the form of a capsule, and between one and four capsules are administered to the patient daily.

19. The method of claim 11, wherein the composition is administered orally in the form of a tablet.

20. The method of claim 11, wherein the composition further comprises a palatability agent, the palatability agent comprising at least one of a plant oil, plant hydrolysates, yeast, and yeast hydrolysates, and combinations thereof.
